(19) United States
(12) Patent Application Publication
(10) Pub. No.: US 2023/0133224 A1
(43) Pub. Date: May 4, 2023

(54) A SWAB AND CASE THEREOF, AND METHODS OF USING THE SAME

(71) Applicants: Daniel SHAHAF, M.P. Emek Ha-Yarden (IL); Eyal BRESSLER, Ramat Gan (IL)

(72) Inventors: Daniel SHAHAF, M.P. Emek Ha-Yarden (IL); Eyal BRESSLER, Ramat Gan (IL)

(21) Appl. No.: 17/918,843

(22) PCT Filed: Apr. 14, 2021
(86) PCT No.: PCT/IL2021/050425

§ 371 Date: Oct. 13, 2022

Related U.S. Application Data
(60) Provisional application No. 63/009,519, filed on Apr. 14, 2020.

(51) Int. Cl. G01N 1/10; B01L 3/00

(52) U.S. Cl. CPC G01N 1/10; B01L 3/5029; A61B 10/0038

(57) ABSTRACT

The present invention discloses, inter alia, swab, kit and a case, disclose a swab as defined above, administrable either or both into and onto a sample in a method comprising steps of providing the pouch-like envelope (1) with the opening (2), the inner surface (1i) and the at least one external surface (1e) with the at last one swabbing portion; inserting a handle into the swabbing portion; applying the swabbing portion either or both onto and into the substance; in-out rolling up the envelope so that the external portion hereto applied either or both onto and into the substance is position within the envelope; wherein at least one of the following s held true: the envelope characterized as a sleeve-like or sock-like elongated member having a main longitudinal axis, configured to be in-out rolled-up along the main longitudinal axis; and the envelope characterized as flexible pouch-like envelope, further characterized by being width-wise squeezable along an axis perpendicular to the main longitudinal axis thereby tightly encapsulated within at least two external case's walls. The present invention discloses a method for both sampling a substance and storing the same, characterized by steps: providing a pouch-like envelope (1), configured to be in-out rolled-up having an opening (2); the pouch comprises an inner surface (1i); and at least one external surface (1e) with at last one swabbing portion (3); the envelope either or both (a) characterized as a sleeve-like or sock-like elongated member having a main longitudinal axis, configured to be in-out rolled-up along the main longitudinal axis; and (b) flexible pouch-like envelope, characterized by being width-wise squeezable along an axis perpendicular to the main longitudinal axis thereby tightly encapsulated within two case's walls; inserting a handle into the swabbing portion; applying the swabbing portion either or both onto and into the substance; in-out rolling up the envelope so that the external portion hereto applied either or both onto and into the substance is position within the envelope; wherein at least one of the following is held true: the envelope characterized as a sleeve-like or sock-like elongated member having a main longitudinal axis, configured to be in-out rolled-up along the main longitudinal axis; and the envelope characterized as flexible pouch-like envelope, further characterized by being width-wise squeezable along an axis perpendicular to the main longitudinal axis thereby tightly encapsulated within at least two external case's walls.

### FIELD OF THE INVENTION

The present invention generally pertains to a swab, kit and case thereof, and to methods of using the same.

### BACKGROUND OF THE INVENTION

Hilal et al. underlines that forensic toxicology is the study and practice of toxicology to the purposes of the law. The relevance of any finding is determined, in the first instance, by the nature and integrity of the specimen(s) submitted for analysis. This means that there are several specific challenges to select and collect specimens for antemortem and post-mortem toxicology investigations; see Hilal, Maha Abd-Elhamed, et al. “Review on Collection, Preservation and Forwarding of Biological Samples for Toxicological Analysis.” Sohag Medical Journal 22.1 (2018): 431-438. Much similarly, U.S. Pat. No. 8,216,832 and EP1692501 related to the field of hazardous material analysis and medical devises and diagnostics thereof. Both patents disclose that art relating to handling of swabs is well established, but remains in need of improvement, both to ensure the integrity of the clinical sample and its protection from contamination, but also to ensure that healthcare professionals are not unnecessarily or inadvertently exposed to biological material on the exterior surfaces of the swab container. Those publications establish that once the external surfaces are contaminated during sample collection, exposure readily occurs when a swab container is passed from hand to hand, and no on-board means is known to refresh or cleanse the outside surfaces of the sample container.

### SUMMARY OF THE INVENTION

It is one object of the invention to disclose a swab for both sampling a substance and storing the same, characterized by pouch-like envelope (e.g., and in a non-limiting manner, see 1 in Figures below). It is in the scope of the invention wherein the envelope either or both (a) characterized as a sleeve-like or sock-like elongated member having a main longitudinal axis, configured to be in-out rolled-up along said main longitudinal axis; and (b) flexible pouch-like envelope, characterized by being width-wise squeezable along an axis perpendicular to the main longitudinal axis thereby tightly encapsulated within two case's walls. The envelope is having at least one opening (2). The pouch comprises an inner surface (1i) and at least one external surface (1e) with at last one swabbing portion (3).

It is yet another object of the invention to disclose a swab as defined above, wherein the swab is administrable either or both into and onto a sample in a method comprising steps of providing the pouch-like envelope (1) with the opening (2), the inner surface (1i) and the at least one external surface (1e) with the at last one swabbing portion; inserting a handle into the swabbing portion; applying the swabbing portion either or both onto and into the substance; and in-out rolling up the envelope so that the external portion hereto applied either or both onto and into the substance is position within the envelope.

It is yet another object of the invention to disclose a swab for both sampling a substance and storing the same, characterized by pouch-like envelope (1), configured to be in-out rolled-up having an opening (2); the pouch comprises an inner surface (1i); and at least one external surface (1e) with at last one swabbing portion (3); the envelope either or both (a) characterized as a sleeve-like or sock-like elongated member having a main longitudinal axis, configured to be in-out rolled-up along the main longitudinal axis; and (b) flexible pouch-like envelope, characterized by being width-wise squeezable along an axis perpendicular to the main longitudinal axis thereby tightly encapsulated within two case's walls.

It is yet another object of the invention to disclose a swab as defined above, administrable either or both into and onto a sample in a method comprising steps of providing the pouch-like envelope (1) with the opening (2), the inner surface (1i) and the at least one external surface (1e) with the at last one swabbing portion; inserting a handle into the swabbing portion; applying the swabbing portion either or both onto and into the substance; in-out rolling up the envelope so that the external portion hereto applied either or both onto and into the substance is position within the envelope; wherein at least one of the following s held true: the envelope characterized as a sleeve-like or sock-like elongated member having a main longitudinal axis, configured to be in-out rolled-up along the main longitudinal axis; and the envelope characterized as flexible pouch-like envelope, further characterized by being width-wise squeezable along an axis perpendicular to the main longitudinal axis thereby tightly encapsulated within at least two external case's walls.

It is yet another object of the invention to disclose a method for both sampling a substance and storing the same, characterized by steps: providing a pouch-like envelope (1), configured to be in-out rolled-up having an opening (2); the pouch comprises an inner surface (1i); and at least one external surface (1e) with at last one swabbing portion (3); the envelope either or both (a) characterized as a sleeve-like or sock-like elongated member having a main longitudinal axis, configured to be in-out rolled-up along the main longitudinal axis; and (b) flexible pouch-like envelope, characterized by being width-wise squeezable along an axis perpendicular to the main longitudinal axis thereby tightly encapsulated within two case's walls; inserting a handle into the swabbing portion; applying the swabbing portion either or both onto and into the substance; in-out rolling up the envelope so that the external portion hereto applied either or both onto and into the substance is position within the envelope; wherein at least one of the following s held true: the envelope characterized as a sleeve-like or sock-like elongated member having a main longitudinal axis, configured to be in-out rolled-up along the main longitudinal axis; and the envelope characterized as flexible pouch-like envelope, further characterized by being width-wise squeezable along an axis perpendicular to the main longitudinal axis thereby tightly encapsulated within at least two external case's walls.

It is yet another object of the invention to disclose a method for both sampling a substance and storing the same by a swab; the method comprising steps of providing a by a sleeve-like envelope (1) with an opening (2), an inner surface (1i) and at least one external surface (1e) with at last one swabbing portion; inserting a handle into the swabbing portion; the handle is selected from a member of group consisting of a hand, at least one finger; a stick, telescoping stick, remote controlled stick, and any combination thereof applying the swabbing portion either or both onto and into the substance; and rolling up a like the envelope like a sock so that the external portion hereto applied either or both onto and into the substance is position within the envelope.

It is yet another object of the invention to disclose a swab for both sampling a substance and storing the same, characterized by a pouch-like envelope (1) with an opening (2); the pouch comprises an inner surface (1i); and at least one external surface (1e) with at last one swabbing portion (3).

It is yet another object of the invention to disclose a swab as defined above, wherein the pouch-like envelope is shaped in a manner selected from a member of group consisting of glove, one or more interconnected sleeves, scabbards, coverings, casings, envelopes, pockets, compartments, sections, layers, sheaths or wrappers; condom; one or more interconnected thimbles and any combination thereof.

It is another object of the invention to disclose a swab as defined in any of the above, wherein the swab is made of, covered by, comprises or otherwise provided in a connection with a composition selected from a member of group consisting of rubber, latex, polymer, cotton, wool, glass-ware, metal-ware, paper-ware such as toilet-paper or any equivalent thereof, and any combination thereof.

It is another object of the invention to disclose a swab as defined in any of the above, wherein the swab is characterized by at least one first open configuration and at least one second closed configuration.

It is another object of the invention to disclose a swab as defined in any of the above, wherein the opening in provided with sealing means (4).

It is another object of the invention to disclose a swab as defined in any of the above, wherein the sealing means (4) are selected from a group consisting of chemical means and physical (mechanical) means.

The swab of claim 6, wherein the chemical sealing means are selected from a group consisting of one or more glue or adhesive.

It is another object of the invention to disclose a swab as defined in any of the above, wherein the physical sealing means are selected from a group consisting of Velcro™, and by-pressing sealing line.

It is another object of the invention to disclose a swab as defined in any of the above, wherein the at last one swabbing portion is characterized by a property selected from a group consisting of texturized surface, adhesive surface, perforated surface, sponge-like surface, continuous and intact surface, smooth surface, hydrophilic/lipophilic balance (HLB)≥1; HLB<1; rigidity, semi-flexibility, flexibility, non-charged surface, negatively charged surface, positively charged surface, zwitterion-charged surface, and any combination thereof.

It is another object of the invention to disclose a swab as defined in any of the above, wherein the at last one swabbing portion is characterized by a property selected from a group consisting of dry surface or wetted surface.

It is another object of the invention to disclose a swab as defined in any of the above, wherein at least one portion of the outer surface is wetted by a member of a group consisting of a fluid.

It is another object of the invention to disclose a swab as defined in any of the above, wherein the fluid is provided useful for one or more of the followings: solvating the substance, to analyze, measure or define it, avoid decontamination of the same and stabilize it.

It is another object of the invention to disclose, in a case, a swab as defined in any of the above.

It is another object of the invention to disclose a case, at least temporarily accommodating a swab as defined in any of the above.

It is another object of the invention to disclose a case as defined above, characterized by at least one first open configuration and at least one second closed configuration.

It is another object of the invention to disclose a case as defined in any of the above, characterized by at least one inner portion (5), at least one swab is irreversibly interconnected with the inner portion of the case.

It is another object of the invention to disclose a case as defined in any of the above, characterized by at least one inner portion (5), at least one swab is reversibly interconnected with the inner portion of the case.

It is another object of the invention to disclose a case as defined in any of the above, wherein the at least one swab interconnects the inner portion of the case at a location being in or adjacent the swab's opening (2).

It is another object of the invention to disclose a case as defined in any of the above, characterized by a locking mechanism configured to lock the case after the swab was used.

It is another object of the invention to disclose diagnostic kit comprising at least one swab as defined in in any of the above

It is another object of the invention to disclose a diagnostic kit comprising at least one swab and its case, as defined in any of the above.

A diagnostic kit as defined in any of the above, further comprising fluids and/or diagnosing-composition.

It is another object of the invention to disclose a method of sampling substance by the swab comprising steps of inserting a handle into a swab; and applying the swab into the substance.

It is another object of the invention to disclose a method as defined above, wherein the handle is selected from a member of group consisting of a hand, at least one finger; an artificial stick, telescoping stick, remote controlled stick and any combination thereof.

It is another object of the invention to disclose a method as defined in any of the above, wherein the handle is attaching to the swab in a means selected from a group consisting of chemical means and physical (mechanical) means.

It is another object of the invention to disclose a method as defined in any of the above, wherein the chemical attach means are provided by one selected from a group consisting of one or more glue or adhesive.

It is another object of the invention to disclose a method as defined in any of the above, wherein the physical attaching means are selected from a group consisting of joints, hinge, external connector, Velcro and a combination thereof.

It is another object of the invention to disclose a method as defined in any of the above, wherein apply the swab into the substance is provided in one of the manners selected from a group consisting of attaching, scratching, peeling, moping and any combination thereof.

It is another object of the invention to disclose a method as defined in any of the above, wherein the substance is a fluid and/or a diagnosing-composition.

It is another object of the invention to disclose a method as defined in any of the above, wherein apply the swab into the substance is provided in one fashion selected from a group consisting of drenching, moping, sponging, soaking; exposing to a substance including aerosol, vapor, gas, steam; and any combination thereof.

It is another object of the invention to disclose a method as defined in any of the above, wherein the swab is at least 50% invert-able from the inside out, in manner to decrease the contamination.

It is another object of the invention to disclose a method of sampling by both the swab and case is given wherein comprising steps of opening the case; inserting a handle into a swab; applying the swab into the substance; introducing the contaminated swab into the case; and sealing the case.

It is another object of the invention to disclose a method as defined in any of the above, wherein the swab is at least 50% invert-able from the inside out, in manner to decrease the contamination.

It is another object of the invention to disclose a method as defined in any of the above, wherein the case content is provided by one preservation material of a group consisting formalin; alcohols; chemicals including ascorbic acid, butylated hydroxytoluene, gallic acid, saline and any combination thereof.

It is another object of the invention to disclose a method as defined in any of the above, wherein the case is either unlockable or lockable.

It is another object of the invention to disclose a method as defined in any of the above, wherein the case is lockable in a manner of one of a group consisting coded lock, key lock, digital lock, mechanical lock including plastic restrain, nylon restrain, cable tie, metal zig and any combination thereof.

It is another object of the invention to disclose a method as defined in any of the above, wherein the case including an ID tags.

It is another object of the invention to disclose a method of sampling by the kit wherein comprising steps of opening the case; inserting a handle into a swab; applying the swab into the substance; covering the contaminated swap by the upper and the lower lid of the case; and sealing the case.

It is another object of the invention to disclose a method as defined in any of the above, wherein the swab is either both connected reversable and irreversible.

It is another object of the invention to disclose a method as defined in any of the above, wherein the swab is at least 50% invert-able from the inside out, in manner to decrease the contamination.

It is another object of the invention to disclose a method of diagnosing by the swab wherein comprising steps of dipping the swab in diagnostic solution; and applying the swab on contaminated substance; and identifying existence of the desire reagent.

It is another object of the invention to disclose a method as defined in any of the above, wherein the diagnostic substance is provided by one of a group consisting enzymatic solution, chemic solution, powder and any combination thereof.

It is another object of the invention to disclose a method of diagnosing by both the swab and case is given wherein comprising steps of applying the swab on contaminated substance; introducing the contaminated swab into the pre-stored diagnostic solution in the case; and identifying existence of the desire reagent;

It is another object of the invention to disclose a method as defined in any of the above, wherein the diagnostic solution stored in a fashion selected of a group consisting ampules, gel-like material, vessel and any combination thereof.

It is another object of the invention to disclose a method as defined in any of the above, wherein the diagnostic substance is provided by one of a group consisting enzymatic solution, chemic solution, powder and any combination thereof.

It is another object of the invention to disclose a wherein method of diagnosing by the kit consisting:

collecting the sample by the swab; folding the case on the contaminated swab; seal the case; and transporting the case to an analytical diagnosis in a laboratory.

It is another object of the invention to disclose a method as defined in any of the above, wherein the analysis performed by one of a group consisting wet chemistry, TLC, see Lan, Gloria, et al. “Stool DNA Testing for Colorectal Cancer Screening in an Urban Medical Center.” Gastroenterology 152.5 (2017): S551 incorporated herein as a reference, HPLC, LPLC, IR-vis-UV spectrometry, ELISA, GCMS, LCMS, XRD, XPS, PCR, RTPCR, Electrophoresis gels.

It is another object of the invention to disclose a method as defined in any of the above, wherein the seal provided by one from a group of rubber stripes, plastic bag, sealing tapes, silicon, nylon and any combination thereof.

It is another object of the invention to disclose a method of transporting by the swab comprising steps of opening the case; inserting a handle into a swab; applying the swab into the substance; inverting the swab inside-out; and transporting the sample.

It is another object of the invention to disclose a method as defined in any of the above, wherein the transport of the contaminated swab provided by one of a group consisting a case, a plastic bag, a nylon bag, a vessel, as part of a kit.

It is another object of the invention to disclose a method as defined in any of the above, wherein the swab is at least 50% inverted inside-out

It is another object of the invention to disclose a method as defined in any of the above, wherein the swab either provided with a self-sealed apparatus or without one.

It is another object of the invention to disclose a method as defined in any of the above, wherein the self-sealed apparatus provided by one from a group of seal strip, removable bag, inflated foam, silicon.

It is another object of the invention to disclose a device useful for both sampling a substance and storing the same, comprising: at least two swabs (10); and a case configured by means of size and shape to securable accommodate the swab(s); wherein the case comprises at least one a first wall portion and at least one second wall portion; a lower cover (22) interconnected to the hinge by an inferior axial (32); and a closing apparatus (23) interconnected to the upper (21) and lower (22) covers; wherein the device is a mobile sealable sampling kit.

It is another object of the invention to disclose the case defined above, wherein the swabs separate by interior wall portion.

It is another object of the invention to disclose the case defined in any of the above, wherein the swabs separated by individual cells.

It is another object of the invention to disclose the case defined in any of the above, wherein the individual cells are both either sealed or unsealed.

It is another object of the invention to disclose the case defined in any of the above, wherein the cells comprising diagnostics.

It is another object of the invention to disclose the case defined in any of the above, wherein the cells comprising each different diagnostic for specific fluids

It is another object of the invention to disclose the case defined in any of the above, wherein the external cover lids are non-stick surfaces made of one from a group consist Teflon, super hydrophobic materials, liquid-impregnated surface

It is another object of the invention to disclose a method of sampling substance by the multi-swab kit wherein comprising steps of intending the material that have to be identified;

choosing, optimizing, or otherwise selecting to the specific swab from the cell that containing the relevant diagnostic solution out of the kit; inserting a handle to the swab; applying the swab on the surface; and identifying existence of the desire reagent.

It is another object of the invention to disclose A swab for both sampling a substance and storing the same, characterized by a flexible pouch-like envelope (1) having a main longitudinal axis AA with an opening (2) located at one end of the axis AA; the pouch comprises an inner surface (1i) with a holding member; at least one external surface (1e) with at last one swabbing portion (3); the flexibility of the envelope is directed to be along or in parallel to the axis AA hence allowing the envelope to be rolled up after use, so that the inner surface (1i) lays externally to the external surface.

It is another object of the invention to disclose the swab as defined above, wherein the pouch-like envelope is shaped in a manner selected from a member of group consisting of glove, one or more interconnected sleeves, scabbards, coverings, casings, envelopes, pockets, compartments, sections, layers, sheaths or wrappers; condom; one or more interconnected thimbles and any combination thereof.

It is another object of the invention to disclose the swab as defined in any of the above, wherein the swab is made of, covered by, comprises or otherwise provided in a connection with a composition selected from a member of group consisting of rubber, latex, polymer, glass-ware, metal-ware, paper-ware, including single layer or multilayered toilet paper, Cellulose-containing products, and equivalents thereof, cotton, wool, and any combination thereof.

It is another object of the invention to disclose the swab as defined in any of the above, wherein the swab is characterized by at least one first open configuration and at least one second closed configuration.

It is another object of the invention to disclose the swab as defined in any of the above, wherein the opening in provided with sealing means (4).

It is another object of the invention to disclose the swab as defined in any of the above, wherein the sealing means (4) are selected from a group consisting of chemical means and physical (mechanical) means.

It is another object of the invention to disclose the swab as defined in any of the above, wherein the chemical sealing means are selected from a group consisting of one or more glue or adhesive.

It is another object of the invention to disclose the swab as defined in any of the above, wherein the physical sealing means are selected from a group consisting of Velcro™, and by-pressing sealing line.

It is another object of the invention to disclose the swab as defined in any of the above, wherein the at last one swabbing portion is characterized by a property selected from a group consisting of texturized surface, adhesive surface, perforated surface, sponge-like surface, continuous and intact surface, smooth surface, hydrophilic/lipophilic balance (HLB)≥1; HLB<1; rigidity, semi-flexibility, flexibility, non-charged surface, negatively charged surface, positively charged surface, zwitterionically charged surface, and any combination thereof.

It is another object of the invention to disclose the swab as defined in any of the above, wherein the at last one swabbing portion is characterized by a property selected from a group consisting of dry surface or wetted surface.

It is another object of the invention to disclose the swab as defined in any of the above, wherein at least one portion of the outer surface is wetted by a member of a group consisting of a fluid.

It is another object of the invention to disclose the swab as defined in any of the above, wherein the fluid is provided useful for one or more of the followings: solvating the substance, to analyze, measure or define it, avoid decontamination of the same and stabilize it.

It is another object of the invention to disclose, in a case, a swab as defined in any of the above.

It is another object of the invention to disclose a case, at least temporarily accommodating a swab as defined in any of the above.

It is another object of the invention to disclose the case as defined in any of the above, wherein it is characterized by at least one first open configuration and at least one second closed configuration.

It is another object of the invention to disclose the case as defined in any of the above, wherein it is characterized by at least one inner portion (5), at least one swab is irreversibly interconnected with the inner portion of the case.

It is another object of the invention to disclose the case as defined in any of the above, wherein it is characterized by at least one inner portion (5), at least one swab is reversibly interconnected with the inner portion of the case.

It is another object of the invention to disclose the case as defined in any of the above, wherein the at least one swab interconnects the inner portion of the case at a location being in or adjacent the swab's opening (2).

It is another object of the invention to disclose the case as defined in any of the above, wherein it is characterized by a locking mechanism configured to lock the case after the swab was used.

It is another object of the invention to disclose an article of manufacture comprising a swab as defined in any of the above.

### BRIEF DESCRIPTION OF THE FIGURES

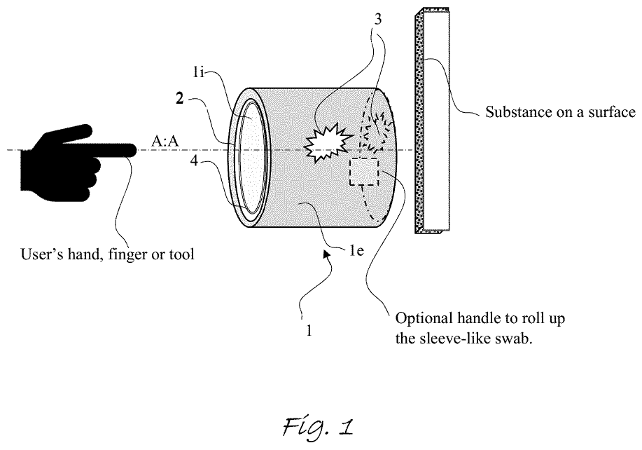

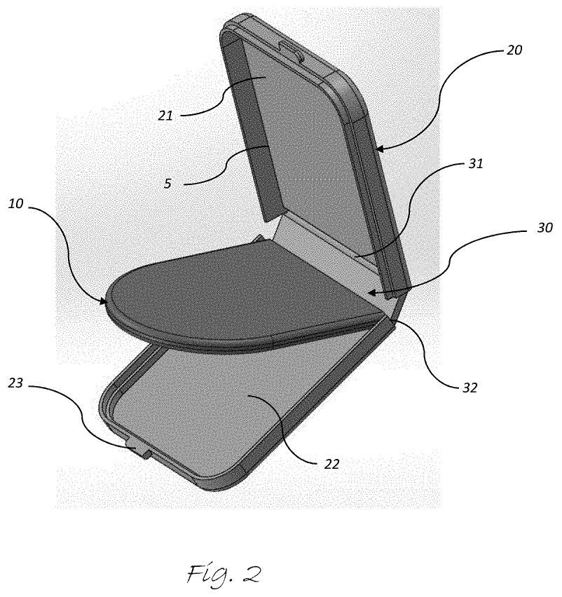

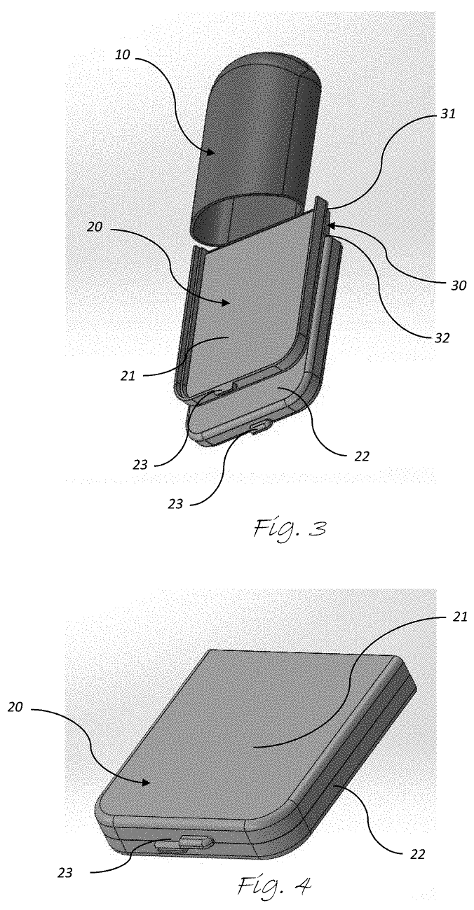

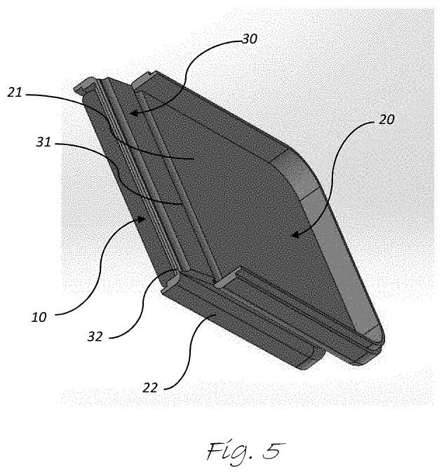

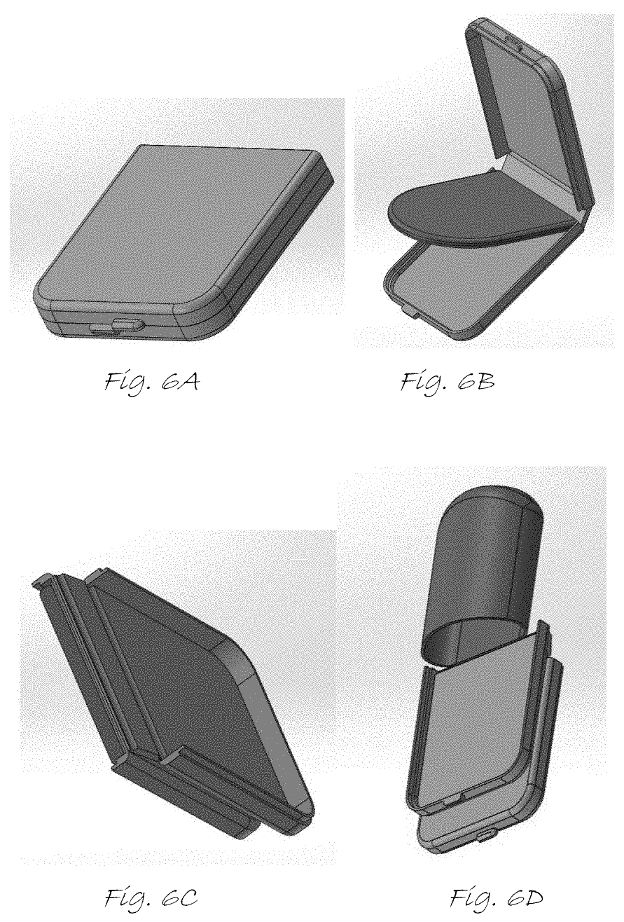

The accompanying drawings, which are included to provide a further understanding of the invention and are incorporated in and constitute a part of this specification, illustrate embodiments of the invention, and together with the description serve to explain the principles of the invention. Each of FIGS. 1, 2, 3, 4, 5a-d depicts an illustration provided here in a non-limiting and out-of-scale manner of an embodiment of the invention.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a swab and case hereof, and to methods of using the same.

As schematically illustrated in FIG. 1, and according to one embodiment of the invention, a first swab for both sampling a substance and storing the same, characterized by a pouch-like envelope (1) with an opening (2) is disclosed. The pouch comprises an inner surface (1i); and at least one external surface (1e) with at last one swabbing portion (3). Here, user's hand or ginger to tool is inserted within the pch-like swab 1 and then the substance to be analyzed is swabbed.

It is well in the scope of the invention wherein the swab comprising swabbing means (e.g., abrasive surfaces etc.) of a first composition; and second type of analyzing-compositions (TLC-related materials). The two type of materials can be either separable, namely provided one next to other by gluing or otherwise connecting; at least partially- or fully-integrated, e.g., embroidered, stitched, hemmed or sewn together. More than two type can be used, e.g., one play (layer) or portion of a layer of a cellulose material is used for analyzing fecal occluded blood, a second play (layer) or portion of this layer of a cellulose material is used for analyzing calprotectin, and thirds play (layer) or portion of a layer of a cellulose material is used for analyzing microbiome-related parameters.

In another embodiment of the invention, a second swab for both sampling a substance and storing the same is disclosed. This swab characterized by a flexible pouch-like envelope (1) having a main longitudinal axis AA with an opening (2) located at one end of the axis AA; the pouch comprises an inner surface (1i) with a holding member; at least one external surface (1e) with at last one swabbing portion (3); the flexibility of the envelope is directed to be along or in parallel to the axis AA hence allowing the envelope to be rolled up after use, so that the inner surface (1i) lays externally to the external surface. In another embodiment of the invention, the opening in provided with sealing means (4).

The term “Adhesive” refers hereinafter in a general manner to any substance that is capable of holding materials together in a functional manner by surface attachment that resists separation. This term is interchangeably used hereinbelow in other terms, including cements, mucilage, naturally or synthetically produced glues, pastes and other applicable terms that are used for any organic material that forms an adhesive bond.

The term is applicable to non-reactive adhesives, e.g., those that harden by drying, which in turn can be solvent-based adhesives, water-based systems and polymer dispersion adhesives (emulsion adhesives), e.g., white glue, contact adhesives and rubber cements, polyvinyl acetate-based cements etc.

The term is also applicable for pressure-sensitive adhesives (PSA) that form a bond by the application of light pressure to marry the adhesive with the adherend. Pressure-sensitive adhesives may be either solids or within liquid carrier, subjected to heating or radiation for cross-linking initiation. PSA include inter alia, acrylate-based polymers, rubber-based, etc., in the presence of a suitable tackifier if needed. The term is also applicable for contact adhesives, solvent-based or water-based, such as natural rubber and polychloroprene (Neoprene).

Hot-melt adhesives, such as ethylene-vinyl acetate-based, polyolefin resins, polyamide, polyester resins and other hot-melts are thermoplastics applied in molten form (in the range of −40° C. to 288° C.) which solidify on cooling to form strong bonds between a wide range of materials.

The term is applicable to chemically reactive adhesives. Hence, the term refers also to anaerobic adhesives that cure when in contact with metal, in the absence of oxygen; multi-part or multi-component adhesives (MCAs), in some cases—pre-mixed and frozen, generally harden by mixing two or more components which chemically react and cross-link into acrylics, urethanes and epoxies-based adhesives. In this general field of solvent-based or solvent-less MCAs, such combinations as polyester-polyurethane resins, polyols-polyurethane resins, acrylic polymers-polyurethane resins are applicable examples.

The term also refers to one-part adhesives that harden via a chemical reaction with an external energy source, such as radiation in the presence or absence of free radical or cationic photo-initiator, electronic beam, heat, and moisture. Hence, acrylic-based and epoxy-based ultra- violet (UV) light curing adhesives, also known as light curing materials (LCM) are applicable. Heat curing adhesives consist of a pre-made mixture of two or more components, e.g., adhesives that include thermoset epoxies, urethanes, and polyimides. Moisture curing adhesives cure when they react with moisture present on the substrate surface or in the air and include e.g., cyanoacrylates, silicones, polyurethanes, polysulfides, one-container type epoxy and urethanes.

The term is further applicable to both natural, semi-synthetic and synthetic adhesives. In a non-limiting manner, natural adhesives are made from organic sources (bio-adhesives) such as vegetables (e.g., starch, dextrin, natural rubber, soybean proteins and vegetable oils), crude oil fractions (asphalt and bitumen), or animals (e.g. the milk protein casein, hide-based animal glues, shellac and chitosan). Starch-based adhesives, casein glue, animal glues derived from collagen hydrolysis, albumen made glues, wood lignin and starch are provided herein as an example. Natural adhesives apply also for inorganic adhesives such as sodium silicate, phosphate and other cements. Semi-synthetic adhesives refer, inter alia, to the cellulosic materials subjected to chemical derivatization to form either water or solvent soluble ethers and esters. To this end, representative examples of semi-synthetic adhesives are nitrate, acetate, butyrate, alkyl and carboxyalkyl cellulose. Synthetic adhesives are also integral members of the aforesaid term. Those adhesives are based on elastomers, thermoplastics, thermosets and alloys. Elastomers are presented by, but not limited to one-part polyurethane, polyisobutylene, nitrile rubber, styrene-butadiene rubber, polysulfide, silicone and chloroprene. Examples of thermosetting adhesives are provided herein on a non-limiting manner, e.g., epoxy, polyurethane, melamine-, phenol-, urea- or resorcinol-formaldehyde, polyesters, silicones, furanes, soluble nylons, polyaromatics and acrylic polymers. Thermoplastic resin adhesives of a synthetic origin include, in a non-limiting manner, polyvinyl esters, acetals, alcohols, alkyl ethers, polystyrene, acrylics, cyanoacrylates, polyamide, polycarbonates, polyacetals, polyethylene and polypropylene and polysulfide. Alloys belong to the named family of synthetic adhesives and are presented by vinyl-, epoxy-, nitrile-phenolics, nylon-, elastomer-, neoprene- and polysulfide-epoxies.

The term “fluid” refers hereinafter to one or more members of a group consisting of one or more gas, one or more liquid, one or more solids and any combination thereof. In another set of embodiments of the invention, the fluids are (i) one or more water miscible compositions;(ii) one or more water immiscible compositions; (iii) one or more surfactants and (iv) a mixture or combination thereof. In another set of embodiments of the invention, the fluids buffers, solvents, cosolvents, emulsifiers, stabilizers, and a mixture or combination thereof.

In another set of embodiments of the invention, the fluids are or otherwise comprise one or more reagent, chemical or biological reactants, immune-diagnosing compositions, genetically-diagnosing compositions, micobiologicals-diagnosing compositions, including pigments colorants, dyes, diagnosing means, anylates, pathogen-diagnosing compositions; hazardous materials--diagnosing compositions; chemical, biological and atomic warfare-diagnosing compositions; clinical or medical-diagnosing such as for the diagnosis of infections, microbiome, cancer, compositions in fecal (occluded blood, see Beg M, Singh M, Saraswat M K, Rewari B B (2002). “Occult Gastrointestinal Bleeding: Detection, Interpretation, and Evaluation” JIACM. 3 (2): 153-58; incorporated herein as a reference, ferritin, viruses, calprotectin etc.), compositions in body fluids and body cavities, and any mixture or combination thereof.

The term “diagnosing compositions” refers hereinafter to comprise one or more reagent, chemical or biological reactants, immune-diagnosing compositions, genetically-diagnosing compositions, micobiologicals-diagnosing compositions, including pigments colorants, dyes, diagnosing means, anylates, pathogen-diagnosing compositions; hazardous materials-diagnosing compositions; chemical, biological and atomic warfare-diagnosing compositions; clinical or medical-diagnosing such as for the diagnosis of infections, microbiome, cancer, compositions in fecal (occluded blood, ferritin, viruses, calprotectin etc.), including Fecal immunochemical testing (FIT), and immunochemical fecal occult blood test (iFOBT) and Stool guaiac test for fecal occult blood (gFOBT), methods and reagents thereof, compositions in body fluids and body cavities, and any mixture or combination thereof.

The term “microbiologicals-diagnosing compositions” refers hereinafter to compositions provided useful or the analysis, diagnostics, measurement of otherwise experimenting microbes, including bacteria, viruses, fungi, protozoa, parasites, and prions. The term also refers to corona viruses, convir19, SARS and others.

In another set of embodiments of the invention, the administrated compositions one or more adhesives. The term “additives” refers hereinafter to one or more members of a group consisting of pharmaceutically acceptable excipients, adjuvants, carriers, antioxidants, preservatives, buffers, biocides, nutrients, minerals and the like. The term also refers to photo-initiators; pigments, dyes, colorants, magnetic substances, leveling agents; wetting agents; adhesion promoters; dispersion aids; anti-blocking agents; anti-caking agents; binders; curing agents; deaerators; diluents; dryers; emulsifiers; fillers; flatting agents; flow control agents; gloss agents; hardeners; lubricants; plasticizers; solvents; stabilizers; surfactants; viscosity modifiers; UV stabilizers; UV absorbers; water repellants, and the like. The term “additives” also refers to stabilizing or enforcing elements, such as ropes, cords, nets, wrappers such as enveloping polymeric sheets, stampers, bands, etc.

The term ‘about’ refers to a value being from 20% less than the defined measure up to 20% more than the defined measure.

As used in the specification and the appended claims, the singular forms “a,” “an” and “the” include their plural referents unless the context clearly dictates otherwise. For example, reference a “polymer,” a “member” or an “additive” is intended to include the processing or making of a plurality of polymers, members or additives. References to a composition containing or including “an” ingredient or “a” polymer is intended to include other ingredients or other polymers, respectively, in addition to the one named.

The terms “comprising” or “containing” or “consisting” or “including” are understood to mean that at least the named compound, element, particle, or method step, etc., is present in the composition or article or method, but does not exclude the presence of other compounds, catalysts, materials, particles, method steps, etc., even if the other such compounds, material, particles, method steps, etc., have the same function as what is named, unless expressly excluded in the claims.

It is also understood that the mention of one or more method steps does not preclude the presence of additional method steps before or after the combined recited steps or intervening method steps between those steps expressly identified. Moreover, the lettering of process steps or ingredients is a convenient means for identifying discrete activities or ingredients and the recited lettering can be arranged in any sequence, unless otherwise indicated.

Numerous modifications and variations in the invention as set forth in the above illustrative examples are expected to occur to those skilled in the art. Consequently, only such limitations as appear in the appended claims should be placed on the invention.

1. A swab for both sampling a substance and storing the same, characterized by pouch-like envelope (1), configured to be in-out rolled-up having an opening (2); said pouch comprises an inner surface (1i); and at least one external surface (1e) with at last one swabbing portion (3); said envelope either or both (a) characterized as a sleeve-like or sock-like elongated member having a main longitudinal axis, configured to be in-out rolled-up along said main longitudinal axis; and (b) flexible pouch-like envelope, characterized by being width-wise squeezable along an axis perpendicular to the said a main longitudinal axis thereby tightly encapsulated within two case's walls

2. The swab of claim 1, said swab is administrable either or both into and onto a sample in a method comprising steps of
a. providing said pouch-like envelope (1) with said opening (2), said inner surface (1i) and said at least one external surface (1e) with said at last one swabbing portion;
b. inserting a handle into said swabbing portion;
c. applying said swabbing portion either or both onto and into the substance; in-out rolling up said envelope so that said external portion hereto applied either or both onto and into the substance is position within said envelope;
wherein at least one of the following s held true:
a. said envelope characterized as a sleeve-like or sock-like elongated member having a main longitudinal axis, configured to be in-out rolled-up along said main longitudinal axis; and
b. said envelope characterized as flexible pouch-like envelope, further characterized by being width-wise squeezable along an axis perpendicular to the said a main longitudinal axis thereby tightly encapsulated within at least two external case's walls.

3. The swab of claim 1, wherein at least one of the following is held true:
a. said handle is selected from a member of group consisting of a hand, at least one finger; a stick, telescoping stick, remote controlled stick, and any combination thereof;
b. said pouch-like envelope is shaped in a manner selected from a member of group consisting of glove, one or more interconnected sleeves, scabbards, coverings, casings, envelopes, pockets, compartments, sections, layers, sheaths or wrappers; condom; one or more interconnected thimbles and any combination thereof;
c. said swab is made of, covered by, comprises or otherwise provided in a connection with a composition selected from a member of group consisting of rubber, latex, polymer, cotton, wool, glass-ware, metal-ware, paper-ware and any combination thereof;
d. said swab is characterized by at least one first open configuration and at least one second closed configuration; and

4. The swab of claim 1, wherein said opening in provided with sealing means (4) and wherein at least one of the following is held true:
a. wherein said sealing means (4) are selected from a group consisting of chemical means and physical (mechanical) means;
b. said chemical sealing means are selected from a group consisting of one or more glue or adhesive; and
c. said physical sealing means are selected from a group consisting of Velcro™, and by-pressing sealing line.

5. The swab of claim 1, wherein said at last one swabbing portion is characterized by a property selected from a group consisting of texturized surface, adhesive surface, perforated surface, sponge-like surface, continuous and intact surface, smooth surface, hydrophilic/lipophilic balance (HLB)≥1; HLB<1; rigidity, semi-flexibility, flexibility, non-charged surface, negatively charged surface, positively charged surface, zwitterion-charged surface, and any combination thereof.

6. The swab of claim 1, wherein said at last one swabbing portion is characterized by a property selected from a group consisting of dry surface or wetted surface.

7. The swab of claim 6, wherein at least one portion of the outer surface is wetted by a member of a group consisting of a fluid.

8. The swab of claim 7, wherein said fluid is provided useful for one or more of the followings: solvating said substance, to analyze, measure or define it, avoid decontamination of the same and stabilize it.

9. In a case, a swab as defined in claim 1 or any of its dependent claims.

10. A case, at least temporarily accommodating a swab as defined in claim 1 or any of its dependent claims.

11. The case of claim 10, characterized by at least one first open configuration and at least one second closed configuration.

12. The case of claim 10, characterized by at least one inner portion (5), at least one swab is irreversibly interconnected with said inner portion of said case.

13. The case of claim 10, characterized by at least one inner portion (5), at least one swab is reversibly interconnected with said inner portion of said case.

14. The case of claim 10 wherein said at least one swab interconnects said inner portion of said case at a location being in or adjacent said swab's opening (2).

15. The case of claim 10, characterized by a locking mechanism configured to lock the case after the swab was used.

16. A diagnostic kit comprising at least one swab as defined in claim 1 or any of its dependent claims; and its case.

17. A diagnostic kit comprising at least one swab and its case, as defined in claim 10 or any of its dependent claims.

18. A diagnostic kit according to any of claim 16 or 17, further comprising fluids and/or diagnosing-composition.

19. A method for both sampling a substance and storing the same, characterized by steps:
a. providing a pouch-like envelope (1), configured to be in-out rolled-up having an opening (2); said pouch comprises an inner surface (1i); and at least one external surface (1e) with at last one swabbing portion (3); said envelope either or both (a) characterized as a sleeve-like or sock-like elongated member having a main longitudinal axis, configured to be in-out rolled-up along said main longitudinal axis; and (b) flexible pouch-like envelope, characterized by being width-wise squeezable along an axis perpendicular to the said a main longitudinal axis thereby tightly encapsulated within two case's walls;
b. inserting a handle into said swabbing portion;
c. applying said swabbing portion either or both onto and into the substance; in-out rolling up said envelope so that said external portion hereto applied either or both onto and into the substance is position within said envelope;
wherein at least one of the following is held true:
a. said envelope characterized as a sleeve-like or sock-like elongated member having a main longitudinal axis, configured to be in-out rolled-up along said main longitudinal axis; and
b. said envelope characterized as flexible pouch-like envelope, further characterized by being width-wise squeezable along an axis perpendicular to the said a main longitudinal axis thereby tightly encapsulated within at least two external case's walls.

20. The method of claim 19, wherein at least one of the following is held true:
a. said handle is selected from a member of group consisting of a hand, at least one finger; a stick, telescoping stick, remote controlled stick, and any combination thereof;
b. said pouch-like envelope is shaped in a manner selected from a member of group consisting of glove, one or more interconnected sleeves, scabbards, coverings, casings, envelopes, pockets, compartments, sections, layers, sheaths or wrappers; condom; one or more interconnected thimbles and any combination thereof;
c. said swab is made of, covered by, comprises or otherwise provided in a connection with a composition selected from a member of group consisting of rubber, latex, polymer, cotton, wool, glass-ware, metal-ware, paper-ware and any combination thereof;
d. said swab is characterized by at least one first open configuration and at least one second closed configuration; and

21. The swab of claim 19, wherein said opening in provided with sealing means (4) and wherein at least one of the following is held true:
a. wherein said sealing means (4) are selected from a group consisting of chemical means and physical (mechanical) means;
b. said chemical sealing means are selected from a group consisting of one or more glue or adhesive; and
c. said physical sealing means are selected from a group consisting of Velcro™, and by-pressing sealing line.

22. The method of claim 19, wherein apply the swab into the substance is provided in one of the manners selected from a group consisting of attaching, scratching, peeling, moping and any combination thereof.

23. The method of claim 19, wherein said substance is a fluid and/or a diagnosing-composition.

24. The method of claim 23, wherein apply the swab into the substance is provided in one fashion selected from a group consisting of drenching, moping, sponging, soaking; exposing to a substance including aerosol, vapor, gas, steam; and any combination thereof.

25. The method of claim 24, wherein said swab is at least 50% invert-able from the inside out, in manner to decrease the contamination.

26. A method of sampling by both said swab and case is given wherein comprising:
a. opening the case; and
b. inserting a handle into a swab; and
c. applying said swab into the substance; and
d. introducing the contaminated swab into the case; and
e. sealing the case.

27. The method of claim 26, wherein said swab is at least 50% invert-able from the inside out, in manner to decrease the contamination.

28. The method of claim 26, wherein said case content is provided by one preservation material of a group consisting formalin; alcohols; chemicals including ascorbic acid, butylated hydroxytoluene, gallic acid, saline and any combination thereof.

29. The method of claim 26, wherein said case is either unlockable or lockable.

30. The method of claim 26, wherein said case is lockable in a manner of one of a group consisting coded lock, key lock, digital lock, mechanical lock including plastic restrain, nylon restrain, cable tie, metal zig and any combination thereof.

31. The method of claim 30, wherein said case including an ID tags.

32. A method of sampling by a kit wherein comprising:
a. opening the case; and
b. inserting a handle into a swab; and
c. applying said swab into the substance; and
d. covering the contaminated swap by the upper and the lower lid of the case; and
e. sealing the case;

33. The method of claim 32, wherein said swab is either both connected reversable and irreversible.

34. The method of claim 32, wherein said swab is at least 50% invert-able from the inside out, in manner to decrease the contamination.

35. A method of diagnosing by a swab wherein comprising:
a. dipping said swab in diagnostic solution; and
b. applying said swab on contaminated substance; and
c. identifying existence of the desire reagent;

36. The method of claim 35, wherein said diagnostic substance is provided by one of a group consisting enzymatic solution, chemic solution, powder and any combination thereof.

37. A method of diagnosing by both a swab and case is given wherein comprising:
a. applying said swab on contaminated substance; and
b. introducing the contaminated swab into the pre-stored diagnostic solution in the case; and
c. identifying existence of the desire reagent;

38. The method of claim 37, wherein said diagnostic solution stored in a fashion selected of a group consisting ampules, gel-like material, vessel and any combination thereof.

39. The method of claim 37, wherein said diagnostic substance is provided by one of a group consisting enzymatic solution, chemic solution, powder and any combination thereof.

40. A method of diagnosing by a kit consisting:
a. collecting the sample by said swab; and
b. folding the case on the contaminated swab; and
c. seal the case; and
d. transporting the case to an analytical diagnosis in a laboratory;

41. The method of claim 40, wherein the analysis performed by one of a group consisting wet chemistry, HPLC, LPLC, IR-vis-UV spectrometry, TLC, ELISA, GCMS, LCMS, XRD, XPS, PCR, RTPCR, Electrophoresis gels.

42. The method of claim 40, wherein said seal provided by one from a group of rubber stripes, plastic bag, sealing tapes, silicon, nylon and any combination thereof.

43. A method of transporting by a swab consisting
a. opening the case; and
b. inserting a handle into a swab; and
c. applying said swab into the substance; and
d. inverting the swab inside-out; and
e. transporting the sample;

44. The method of claim 43, wherein the transport of the contaminated swab provided by one of a group consisting a case, a plastic bag, a nylon bag, a vessel, as part of a kit.

45. The method of claim 43, wherein said swab is at least 50% inverted inside-out

46. The method of claim 43, wherein said swab either provided with a self-sealed apparatus or without one.

47. The method of claim 46, wherein said self-sealed apparatus provided by one from a group of seal strip, removable bag, inflated foam, silicon.

48. A device useful for both sampling a substance and storing the same, comprising:
a. at least two swabs (10); and
b. a case configured by means of size and shape to securable accommodate said swab(s); wherein said case comprises at least one a first wall portion and at least one second wall portion;
c. a lower cover (22) interconnected to said hinge by an inferior axial (32); and
d. a closing apparatus (23) interconnected to said upper (21) and lower (22) cove;
wherein said device is a mobile sealable sampling kit.

49. The case of claim 48, wherein at least one of the following is held true:
a. said swabs separate by interior wall portion;
b. said swabs separated by individual cells;
c. said individual cells are both either sealed or unsealed;
d. said cells comprising diagnostics;
e. said cells comprising each different diagnostic for specific fluids; and
f said external cover lids are non-stick surfaces made of one from a group consist Teflon, super hydrophobic materials, liquid-impregnated surface.

50. A method of sampling substance by a multi-swab kit wherein comprising:
a. intending the material that have to be identified;
b. choosing to the specific swab from the cell that containing the relevant diagnostic solution out of the kit;
c. insert a handle to the swab;
d. applying the swab on the surface; and
e. identifying existence of the desire reagent.

51. A swab for both sampling a substance and storing the same, characterized by a flexible pouch-like envelope (1) having a main longitudinal axis AA with an opening (2) located at one end of said axis AA; said pouch comprises
a. an inner surface (1i) with a holding member;
b. at least one external surface (1e) with at last one swabbing portion (3); and
c. said flexibility of said envelope is directed to be along or in parallel to said axis AA hence allowing said envelope to be rolled up after use, so that said inner surface (1i) lays externally to said external surface.

52. The swab of claim 51 wherein at least one of eth following is held true:
a. said pouch-like envelope is shaped in a manner selected from a member of group consisting of glove, one or more interconnected sleeves, scabbards, coverings, casings, envelopes, pockets, compartments, sections, layers, sheaths or wrappers; condom; one or more interconnected thimbles and any combination thereof;
b. said swab is made of, covered by, comprises or otherwise provided in a connection with a composition selected from a member of group consisting of rubber, latex, polymer, glass-ware, metal-ware, paper-ware, cotton, wool, and any combination thereof;
c. said swab is characterized by at least one first open configuration and at least one second closed configuration;
d. said opening in provided with sealing means (4);
e. sealing means (4) are selected from a group consisting of chemical means and physical (mechanical) means;
f. said chemical sealing means are selected from a group consisting of one or more glue or adhesive;
g. said physical sealing means are selected from a group consisting of Velcro', and by-pressing sealing line;
h. said at last one swabbing portion is characterized by a property selected from a group consisting of texturized surface, adhesive surface, perforated surface, sponge-like surface, continuous and intact surface, smooth surface, hydrophilic/lipophilic balance (HLB)≥1; HLB<1; rigidity, semi-flexibility, flexibility, non-charged surface, negatively charged surface, positively charged surface, zwitterion-charged surface, and any combination thereof;
i. said at last one swabbing portion is characterized by a property selected from a group consisting of dry surface or wetted surface;
j. at least one portion of the outer surface is wetted by a member of a group consisting of a fluid;
k. at least one portion of the outer surface is wetted by a member of a group consisting of a fluid and said fluid is provided useful for one or more of the followings: solvating said substance, to analyze, measure or define it, avoid decontamination of the same and stabilize it.

53. In a case, a swab as defined in claim 51 or any of its dependent claims.

54. A case, at least temporarily accommodating a swab as defined in claim 51 or any of its dependent claims.

55. The case of claim 51, wherein at least one of the following is held true:
a. said case is characterized by at least one first open configuration and at least one second closed configuration;
b. said case is characterized by at least one inner portion (5), at least one swab is irreversibly interconnected with said inner portion of said case;
c. said case is characterized by at least one inner portion (5), at least one swab is reversibly interconnected with said inner portion of said case.;
d. said at least one swab interconnects said inner portion of said case at a location being in or adjacent said swab's opening (2);
e. said case is characterized by a locking mechanism configured to lock the case after the swab was used.

56. A diagnostic kit comprising at least one swab as defined in claim 51 or any of its dependent claims; and its case.
